(19) United States
(12) Patent Application Publication
(10) Pub. No.: US 2025/0017536 A1
(43) Pub. Date: Jan. 16, 2025

(54) CHRONIC OBSTRUCTIVE PULMONARY DISEASE MONITORING DEVICE

(71) Applicant: Medtronic, Inc., Minneapolis, MN (US)

(72) Inventor: Geert Morren, Vissenaken (BE)

(21) Appl. No.: 18/707,353

(22) PCT Filed: Nov. 2, 2022
(86) PCT No.: PCT/IB2022/060566

§ 371 Date: May 3, 2024

Related U.S. Application Data
(60) Provisional application No. 63/264,742, filed on Dec. 1, 2021.

(51) Int. Cl. A61B 5/00; A61B 5/01; A61B 5/318

(52) U.S. Cl. CPC A61B 5/7275; A61B 5/01; A61B 5/318

(57) ABSTRACT

An example device includes memory configured to store an N-dimensional model, where N is equal to a count of a plurality of physiological parameters and processing circuitry communicatively coupled to the memory. The processing circuitry is configured to determine a plurality of signals indicative of physical quantities of the patient and to process the plurality of signals indicative of physical quantities of the patient to determine a plurality of physiological parameters of the patient. The processing circuitry is configured to process the plurality of physiological parameters of the patient to the N-dimensional model to determine a chronic obstructive pulmonary disease (COPD)-exacerbation risk score and determine whether the COPD-exacerbation risk score satisfies a threshold. The processing circuitry is configured to, generate an indication for output that is based at least in part on the COPD-exacerbation risk score satisfying the threshold.

### FIELD

This disclosure generally relates to medical devices and, more particularly, analysis of signals sensed by medical devices.

### BACKGROUND

Medical devices may be used to monitor physiological parameters of a patient. For example, some medical devices are configured to sense cardiac electrogram (EGM) signals, e.g., electrocardiogram (ECG) signals, indicative of the electrical activity of the heart via electrodes.

Chronic obstructive pulmonary disease (COPD) is a disease similar to heart disease in certain aspects. COPD is a chronic and progressive disorder. When a patient is hospitalized for COPD, the health of the patient does not normally return to the level that the health of the patient was prior to the hospitalization. When symptoms are stable, a person with COPD may use an inhaler, for example, twice a day. In many COPD patients, the symptoms will acutely worsen from time to time, mostly triggered by an infection in the lungs or airways. If a COPD patient has a virus, for example, the patient may get very short of breath and their oxygen saturation may decrease dramatically. To alleviate such acute worsening (also called a “COPD exacerbation”), a clinician may increase the doses of the medication, administer additional medication (such as steroids or antibiotics), or provide oxygen to such a patient. For example, if the patient is coughing more, the clinician may advise the patient to use the inhaler more often or prescribe another kind of inhaler. The changes in treatment to alleviate an exacerbation, however, are often not provided soon enough.

### SUMMARY

COPD is progressive lung disease characterized by persistent airflow limitation and a gradual deterioration in lung function with multiple distressing symptoms, such as dyspnea (e.g., breathlessness). Another characteristic of COPD is acute exacerbations, defined as an acute worsening of the respiratory symptoms (dyspnea, coughing, and/or sputum), which may require additional therapy. Such exacerbations may typically last for several days and often result in hospitalizations. Because exacerbations strongly affect quality of life of someone suffering from COPD, disease progression of COPD, and health care costs, it may be desirable to detect such exacerbations early, so that treatment can be started promptly, thereby reducing the risk of hospitalization, and improving quality-of-life of a patient suffering from COPD.

Although many COPD patients are educated on how to assess their own symptoms, exacerbations may often be detected too late by the patient. This is because the symptoms are mostly assessed subjectively (e.g., using questionnaires), and it may be difficult for a patient to assess whether the worsening of the symptom(s) is beyond normal day-to-day variability. Dyspnea, which may be the main symptom of COPD, for example, is typically assessed using patient questionnaires that are designed to relate the severity of dyspnea with the associated level of activity/exertion, as dyspnea is typically most pronounced upon exertion.

In general, this disclosure describes techniques for monitoring for disease exacerbations in a COPD patient. An IMD may determine a plurality of signals indicative of physical parameters multiple times per day, under different conditions, such as different levels of activity or exertion. In some examples, the IMD may determine physiological parameters related to the COPD symptoms from the plurality of signals and determine deviations of the physiological parameters from a baseline state to determine exacerbations in the COPD patient. A medical device, such as an implantable medical device or an insertable medical device may determine a number (M) of signals indicative of physical quantities of the COPD patient and may process the determined physical properties to determine a number (N) of physiological parameters of the patient. The medical device may make such determinations several times a day. The medical device may process the determined physiological parameters of the patient to an N-dimensional model to determine a COPD-exacerbation risk score. For example, the medical device may compare the determined physiological parameters of the patient to the N-dimensional model to determine the COPD-exacerbation risk score. If the COPD-exacerbation risk score satisfies a threshold, the medical device may generate an indication for output based at least in part on the COPD-exacerbation risk score satisfying the threshold. For example, the medical device may generate an alert based on the COPD-exacerbation risk score satisfying the threshold and output the alert to an external device. Such an alert may facilitate the patient to seek medical treatment, or a clinician to administer additional or alternative medication and/or treatment earlier than otherwise may be. This early alert regarding an exacerbation of the COPD symptoms may guide medical treatment of the patient, avoid or shorten hospitalization stays, and result in a better state of health of the patient than if the patient were to subjectively attempt to determine when the disease may be exacerbated.

In some examples, a medical device system comprises memory configured to store an N-dimensional model, where N is equal to a count of a plurality of physiological parameters; and processing circuitry communicatively coupled to the memory, the processing circuitry being configured to: determine a plurality of signals indicative of physical quantities of the patient; process the plurality of signals indicative of physical quantities of the patient to determine the plurality of physiological parameters of the patient; process the plurality of physiological parameters of the patient to the N-dimensional model to determine a chronic obstructive pulmonary disease (COPD)-exacerbation risk score; determine whether the COPD-exacerbation risk score satisfies a threshold; and generate an indication for output that is based at least in part on the COPD-exacerbation risk score satisfying the threshold.

In some examples, a method of monitoring a patient comprises: determining, by a medical device, a plurality of signals indicative of physical quantities of the patient; processing, by the processing circuitry, the plurality of signals indicative of physical quantities of the patient to determine a plurality of physiological parameters of the patient; processing, by the processing circuitry, the plurality of physiological parameters of the patient to an N-dimensional model, where N is equal to a count of the plurality of physiological parameters, to determine a chronic obstructive pulmonary disease (COPD)-exacerbation risk score; determining, by the processing circuitry, whether the COPD-exacerbation risk score satisfies a threshold; and generating, by the processing circuitry, an indication for output that is based at least in part on the COPD-exacerbation risk score satisfying the threshold.

In some examples, a computer-readable medium comprising instructions, which, when executed, cause processing circuitry to: determine a plurality of signals indicative of physical quantities of a patient; process the plurality of signals indicative of physical quantities of the patient to determine a plurality of physiological parameters of the patient; process the plurality of physiological parameters of the patient to the N-dimensional model to determine a chronic obstructive pulmonary disease (COPD)-exacerbation risk score, where N is equal to a count of the plurality of physiological parameters; determine whether the COPD-exacerbation risk score satisfies a threshold; and generate an indication for output that is based at least in part on the COPD-exacerbation risk score satisfying the threshold.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example of a medical device system configured to monitor physiological parameters of a patient in accordance with the techniques of the disclosure.

FIG. 2 is a block diagram illustrating an example configuration of the implantable medical device (IMD) of FIG. 1.

FIG. 3 is a conceptual side-view diagram illustrating an example configuration of the IMD of FIGS. 1 and 2.

FIG. 4 is a functional block diagram illustrating an example configuration of external device.

FIG. 5 is a graphical diagram illustrating an example of health status of a patient over time.

FIG. 6 is a graphical diagram illustrating an example of 2 physiological parameters.

FIG. 7 is a block diagram illustrating example signals indicative of physical quantities, processing, and physiological parameters which may be used to determine an exacerbation.

FIG. 8 is a flow diagram illustrating example COPD monitoring techniques of this disclosure.

FIG. 9 is a flow diagram illustrating an additional example of COPD monitoring techniques of this disclosure.

Like reference characters refer to like elements throughout the figures and description.

### DETAILED DESCRIPTION

A variety of types of implantable or insertable medical devices monitor physiological parameters of a patient. Implantable medical devices (IMDs) can sense and monitor cardiac EGMs, and detect arrhythmia episodes. Example IMDs that monitor cardiac electrograms (EGMs) include pacemakers and implantable cardioverter-defibrillators, which may be coupled to intravascular or extravascular leads, as well as pacemakers with housings configured for implantation within the heart, which may be leadless. Some IMDs that do not provide therapy, e.g., implantable patient monitors, sense cardiac EGMs. One example of such an IMD is the Reveal LINQ™ Insertable Cardiac Monitor (ICM), available from Medtronic plc, which may be inserted subcutaneously. Such IMDs may facilitate relatively longer-term monitoring of patients during normal daily activities, and may periodically transmit collected data or alerts to external devices or to a patient monitoring service, such as the Medtronic Carelink™ Network.

In some examples, an IMD may be used to monitor a COPD patient for an acute COPD exacerbation event. By objectively determining an acute exacerbation event and generating an indication for output, for example, an alert for output to an external device, the IMD may facilitate the early treatment of the exacerbation, thereby preserving as much of the health status of the patient as possible.

This disclosure describes a medical device system that uses processing circuitry to determine a high risk of an acute exacerbation event in a COPD patient and output an alert to an external device. The medical device system may include a medical device, such as one of the devices described above or any other type of implantable device, such as a subcutaneous cardiac monitoring device, a single chamber ICD, an extravascular ICD, a subcutaneous ICD, or any other type of device configured to monitor physiological parameters of a patient.

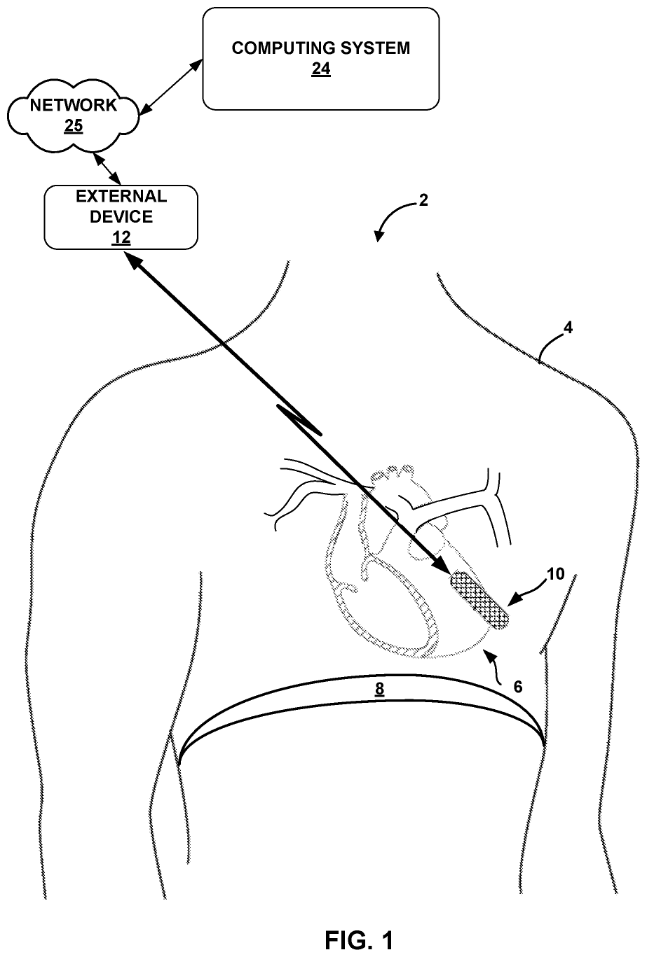

FIG. 1 is a conceptual drawing illustrating an example of a medical device system configured to monitor physiological parameters of a patient in accordance with the techniques of the disclosure. While primarily described herein as monitoring a COPD patient, in some examples, IMD 10 may monitor other patients having other diseases or disorders. Additionally, the techniques described herein as being performed by IMD 10 may be performed by other implantable or insertable medical devices.

The example techniques may be used with IMD 10, which may be in wireless communication with an external device 12. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, ID 10 is positioned near the respiratory muscles (e.g., diaphragm 8, parasternal muscles, or intercostal muscles) of patient 4. IMD 10 includes a plurality of electrodes (not shown in FIG. 1), and may be configured to sense or measure any combination of a cardiac EGM (e.g., an ECG) via the plurality of electrodes, accelerometer signals, an EMG via the plurality of electrodes, impedance of tissue via the plurality of electrodes, optical sensor signals, temperature sensor signals, or other signals indicative of a physical quantity of patient 4. In some examples, IMD 10 takes the form of the LINQ™ ICM. Although described primarily in the context of examples in which the medical device that monitors for an acute exacerbation event takes the form of an ICM, the techniques of this disclosure may be implemented in systems including any one or more implantable or external medical devices, including monitors, pacemakers, defibrillators, or other devices configured to monitor physiological parameters of a patient.

External device 12 is a computing device configured for wireless communication with IMD 10. External device 12 may be, as examples, a mobile telephone or other computing device of patient 4 or another user, or a computing device configured to communicate with IMP 10. External device 12 may be configured to communicate with computing system 24 via network 25. In some examples, external device 12 may provide a user interface and allow a user to interact with IMD 10. Computing system 24 may comprise computing devices configured to allow a user to interact with IMP 10, or data collected from IMP, via network 25.

External device 12 may be used to receive or retrieve data from IMP 10 and may transmit the data to computing system 24 via network 25. The retrieved data may include values of physiological parameters measured by IMP 10, indications of COPD exacerbations, or other maladies detected by IMD 10, and other physiological signals recorded by IMD 10.

In some examples, computing system 24 includes one or more handheld computing devices, computer workstations, servers or other networked computing devices. In some examples, computing system 24 may include one or more devices, including processing circuitry and storage devices, that implement a monitoring system 450. Computing system 24, network 25, and monitoring system 450 may be implemented by the Medtronic Carelink™ Network or other patient monitoring system, in some examples.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as computing system 24 and IMD 10, access to the Internet, and may provide a communication framework that allows the computing devices to communicate with one another. In some examples, network 25 may be a private network that provides a communication framework that allows computing system 24, IMD 10, and/or external device 12 to communicate with one another but isolates one or more of computing system 24, IMD 10, or external device 12 from devices external to network 25 for security purposes. In some examples, the communications between computing system 24, IMD 10, and external device 12 are encrypted.

Processing circuitry of medical device system 2 (e.g., of IMD 10, external device 12, computing system 24, and/or of one or more other computing devices) may be configured to perform the example techniques of this disclosure for monitoring for and determining an exacerbation of COPD symptoms in patient 4.

IMD 10 may be configured to monitor a plurality of physiological parameters of patient 4 which IMD 10 may determine from a plurality of signals indicative of physical quantities. ID 10 may build or create a probability distribution of determined physiological parameters (N-dimensional) over a baseline time period of a certain duration. In some examples, this baseline time period may be in the range of one week to one year, for example, one month. In some examples, if the patient is hospitalized, a new baseline time period may be used to build or create a probability distribution of determined physiological parameters (N-dimensional), for example, starting from a time after hospitalization. After the baseline period, for each new determined set of physiological parameters, IMD 10 may calculate the probability that this new set of parameters belongs to the probability distribution of the preceding period or the baseline period or is outside of the probability distribution of the preceding period or the baseline period. Based on the probabilities of the last set of physiological parameters, IMD 10 may, determine a COPD-exacerbation risk score for exacerbation and, when the COPD-exacerbation risk score satisfies an exacerbation threshold (also referred to herein as a threshold), IMD 10 may transmit an alert to external device 12 which may provide a warning for the patient, caregiver, and/or clinician. For example, the COPD-exacerbation risk score may satisfy the exacerbation threshold by being greater than, greater than or equal to, equal to, less than, or less than or equal to the exacerbation threshold. By transmitting the alert, IMD 10 may facilitate more speedy treatment of the patient by the clinician than otherwise may occur, thereby preserving health of patient 4.

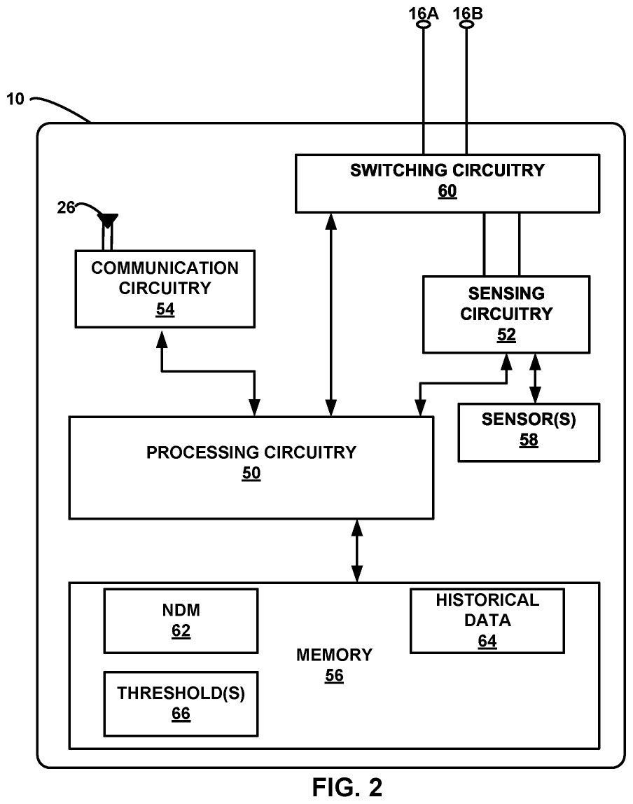

For example, IMD 10 may monitor an electromyogram (EMG) of respiratory muscles, as objective measure of dyspnea. ID 10 may determine an impedance of tissue, such as respiratory muscles, subcutaneous tissue surrounding ID 10, or other tissue, and may determine a respiration rate and/or changes in tidal volume based on the determined impedance. The respiration rate and/or changes in tidal volume may be used as a measure of exertion. ID 10 may include an accelerometer (e.g., one of sensor(s) 58 of FIG. 2). ID 10 may determine activity levels, posture, respiration rate, and/or tidal volume based on one or more signals from the accelerometer. In some examples, IMD 10 may additionally determine ventilation from the determined respiration rate and the determined tidal volume. IMD 10 may measure an ECG and determine a heart rate from the ECG. IMD 10 may sense a temperature of patient 4. IMD 10 may also sense oxygen saturation of patient 4, for example, through an optical sensor (e.g., of sensor(s) 58) such as a pulse oximeter.

The techniques of this disclosure may include collecting objective data on COPD symptoms multiple times during daily life under various conditions. Such objective data may be used to determine an exacerbation of COPD symptoms more reliably than a subjective opinion of patient 4. Therefore, IMD 10 may detect an exacerbation of COPD symptoms earlier than patient 4 may, allowing the patient, caregiver, or clinician to start treatment of the patient earlier than otherwise which may avoid the need for hospitalization of patient 4 or reduce the length of hospitalization, while leading to less degradation of the health of patient 4.

FIG. 2 is a block diagram illustrating an example configuration of IMD 10 of FIG. 1. As shown in FIG. 2, IMD 10 includes processing circuitry 50, sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media. Memory 56 may also store an N-dimensional model (NDM) 62 and historical data 64. N-dimensional model 62 may be an N-dimensional probability distribution function, an N-dimensional machine learning model including a neural network, or the like. Historical data 64 may include determined signals indicative of physical quantities of patient 4, determined physiological parameters of patient 4, and/or other data related to patient 4 or treatment of patient 4. Memory 56 may also store threshold 66 which may be used to determine an exacerbation of COPD symptoms in patient 4.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor various physiological parameters of patient 4 of FIG. 1. Processing circuitry 50 may generate an indication for output if the physiological parameters indicate an exacerbation of COPD symptoms for patient 4. For example, processing circuitry 50 may control communication circuitry 54 to output an alert (e.g., to external device 12). In some examples, processing circuitry 50 may control sensing circuitry 52 and or sensors 58 (which may include an accelerometer, an optical sensor, or a temperature sensor) to sense or determine a plurality of signals indicative of physical quantities. Processing circuitry 50 may process the plurality of signals indicative of physical quantities to determine a plurality of physiological parameters of patient 4. Processing circuitry 50 may process the plurality of physiological parameters of the patient to an N-dimensional model 62, where N is equal to a count of the plurality of physiological parameters, to determine a COPD-exacerbation risk score. For example, processing circuitry 50 may compare the plurality of physiological parameters of the patient to an N-dimensional model 62 to determine a COPD-exacerbation risk score. Processing circuitry 50 may determine whether the COPD-exacerbation risk score satisfies a threshold and, generate an indication that is based at least in part on the COPD-exacerbation risk score satisfying the threshold. For example, processing circuitry 50 may control communication circuitry 54 to output an alert to external device 12. Such an alert may be indicative of patient 4 having exacerbated COPD symptoms. In some examples, the alert may include the determined physiological parameters, so as to guide treatment of patient 4 by a clinician. The physician may further assess the (individual) physiological parameters of an “abnormal” set(s) of N physiological parameters (e.g., those set(s) for which the COPD-exacerbation risk score satisfies the threshold). For example, if dyspnea has worsened, a clinician may prescribe a different inhaler or steroids for patient 4.

In some examples, processing circuitry 50 may store sensed or measured signals indicative of physical quantities in memory 56. In some examples, processing circuitry may store determined physiological parameters in memory 56. In some examples, processing circuitry may store a determined COPD-exacerbation risk score in memory 56.

Sensing circuitry 52 and/or processing circuitry 50 may be configured to process signals indicative of physical quantities to determine the physiological parameters and may include filters, peak detectors, envelope calculations, in some examples.

In some examples, IMD 10 includes one or more sensors 58, such as one or more accelerometers, microphones, optical sensors, temperature sensors, pressure sensors and/or other sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 4 based on signals from sensors 58, which may be used to determine an exacerbation of COPD symptoms in patient 4.

Communication circuitry 54 may include any suitable hardware, firmware, software or any combination thereof for communicating with another device, such as external device 12. Communication circuitry 54 may be configured to communicate using any of a variety of wireless communication schemes, such as Bluetooth® or Bluetooth Low Energy®. Under the control of processing circuitry 50, communication circuitry 54 may receive downlink telemetry from, as well as send uplink telemetry to, external device 12 or another device with the aid of an internal or external antenna, e.g., antenna 26. In some examples, processing circuitry 50 may communicate with a networked computing device (e.g., computing system 24) via an external device (e.g., external device 12) and a computer network, such as the Medtronic CareLink® Network developed by Medtronic, plc, of Dublin, Ireland.

Although described herein in the context of example IMD 10, the techniques for detecting exacerbation of COPD symptoms disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a medical device external to patient 4, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, or a “smart” watch.

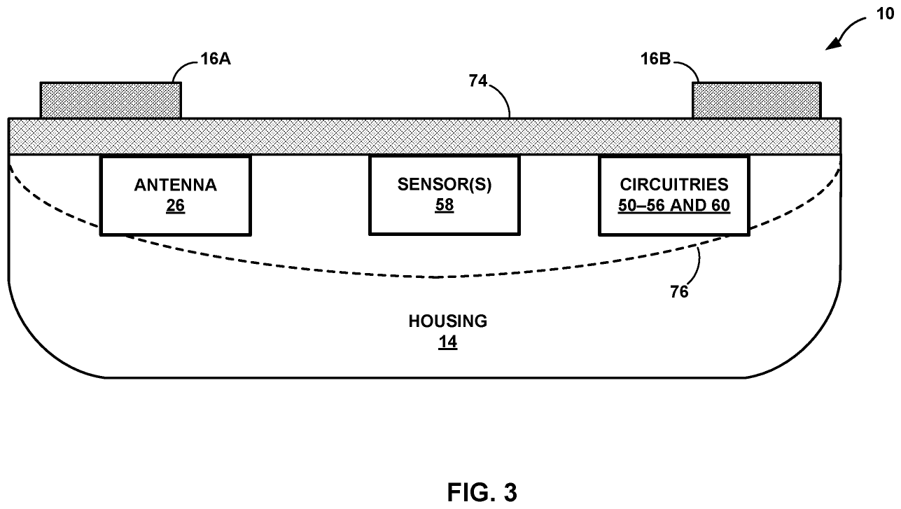

FIG. 3 is a conceptual side-view diagram illustrating an example configuration of IMD 10. In the example shown in FIG. 3, IMD 10 may include a leadless, subcutaneously-implantable monitoring device having a housing 14 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 14. In the illustrated example, antenna 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 14 such that housing 14 and cover 74 enclose antenna 26, sensors 58, and circuitries 50-56 and 60, and protect the antenna and circuitries from fluids such as body fluids.

One or more of antenna 26, sensors 58, or circuitries 50-56 may be formed on insulative cover 74, such as by using flip-chip technology. Insulative cover 74 may be flipped onto a housing 14. When flipped and placed onto housing 14, the components of IMD 10 formed on the inner side of insulative cover 74 may be positioned in a gap 76 defined by housing 14. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

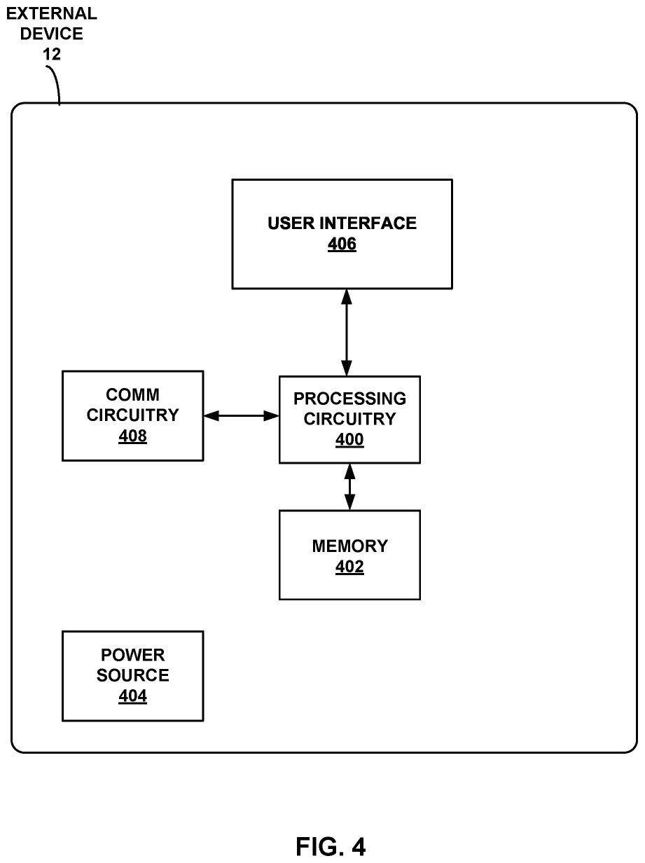

FIG. 4 is a functional block diagram illustrating an example configuration of external device. External device 12 may include processing circuitry 400, memory 402, communication circuitry 408, user interface 406, and power source 404. Processing circuitry 400 controls user interface 406 and communication circuitry 408, and stores and retrieves information and instructions to and from memory 402. External device 21 may be configured for use as a clinician programmer or a patient programmer. Processing circuitry 400 may include any combination of one or more processors including one or more microprocessors, DSPs, ASICs, FPGAs, or other equivalent integrated or discrete logic circuitry. Accordingly, processing circuitry 400 may include any suitable structure, whether in hardware, software, firmware, or any combination thereof, to perform the functions ascribed herein to processing circuitry 400.

A user, such as a clinician or patient 114, may interact with external device 12 through user interface 406. User interface 406 may include a display, such as an LCD or LED display or other type of screen, one or more speakers, a haptic device, or other devices to present information. In some examples, user interface 406 may be configured to alert patient 4, a caregiver, or a clinician of an acute exacerbation of COPD symptoms in patient 4, such that the clinician may take the appropriate measures to treat patient 4 earlier than if patient 4 were subjectively monitoring their own systems. For example, such an alert may be visual, auditory, and/or haptic. In some examples, the alert may include the determined physiological parameters, so as to guide treatment of patient 4 by a clinician. In addition, user interface 406 may include an input mechanism to receive input from the user. The input mechanisms may include, for example, buttons, a keypad (e.g., an alphanumeric keypad), a peripheral pointing device, one or more microphones, or another input mechanism that allows the user to navigate through user interfaces presented by processing circuitry 400 of external device 21 and provide input.

In some examples, at least some of the techniques described herein may be implemented by processing circuitry 400 of external device 12. For example, processing circuitry 400 may determine a COPD-exacerbation risk score, generate an indication for output, and output an alert via user interface 406 regarding an acute exacerbation event based on information provided by IMD 10.

Memory 402 may include instructions for operating user interface 406 and communication circuitry 408, and for managing power source 404. Memory 402 may also store any data retrieved from IMD 10. The clinician may use this data to determine the progression of the patient condition in order to determine future treatment. Memory 402 may include any volatile or nonvolatile memory, such as RAM, ROM, EEPROM or flash memory. Memory 402 may also include a removable memory portion that may be used to provide memory updates or increases in memory capacities. A removable memory may also allow sensitive patient data to be removed before external device 12 is used by a different patient.

Wireless telemetry in external device 12 may be accomplished by use of communication circuitry 408, which may communicate with a proprietary protocol or industry-standard protocol such as using the Bluetooth® specification set. Accordingly, communication circuitry 408 may be similar to the communication circuitry contained within by IMD 10. In alternative examples, external device 12 may be capable of infrared communication or direct communication through a wired connection. In this manner, other external devices may be capable of communicating with external device 12 without needing to establish a secure wireless connection.

Power source 404 may deliver operating power to the components of external device 12. Power source 404 may include a battery and a power generation circuit to produce the operating power. In some examples, the battery may be rechargeable to allow extended operation. Recharging may be accomplished by electrically coupling power source 404 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate. Power source 404 may include circuitry to monitor power remaining within a battery. In this manner, user interface 406 may provide a current battery level indicator or low battery level indicator when the battery needs to be replaced or recharged. In some cases, power source 404 may be capable of estimating the remaining time of operation using the current battery.

According to the techniques of this disclosure, external device 12 may be used to receive from IMD 10 an alert regarding an acute exacerbation of COPD symptoms in patient 4. For example, external device 12 may provide such indications to a clinician or other device to help guide treatment of patient 4.

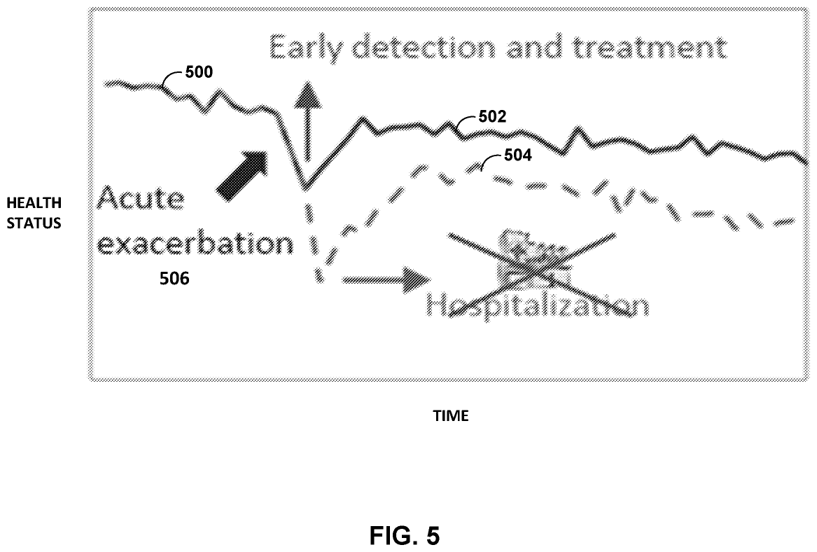

FIG. 5 is a graphical diagram illustrating an example of health status of a patient over time. In the example of FIG. 5, a health status of a patient (e.g., patient 4 of FIG. 1) is depicted as line 500 prior to acute exacerbation event 506. Because IMD 10 may more effectively and objectively determine an exacerbation event is occurring than patient 4 subjectively attempting to determine whether day-to-day variability of symptoms is an acute exacerbation event, and IMD 10 may generate an indication for output. For example, IMD 10 may generate and output an alert, e.g., to external device 12, patient 4 may receive treatment for the exacerbation earlier than patient 4 may otherwise receive. Thus, patient 4 may come closer to completely recovering from the exacerbation as shown in line 502. If patient 4 were to attempt to subjectively determine an acute exacerbation without IMD 10, the patient may require hospitalization or longer hospitalization because of a delay in seeking or receiving treatment. This may result in a deeper exacerbation of COPD symptoms and less of a return to a health status prior to the acute exacerbation, as shown with line 504.

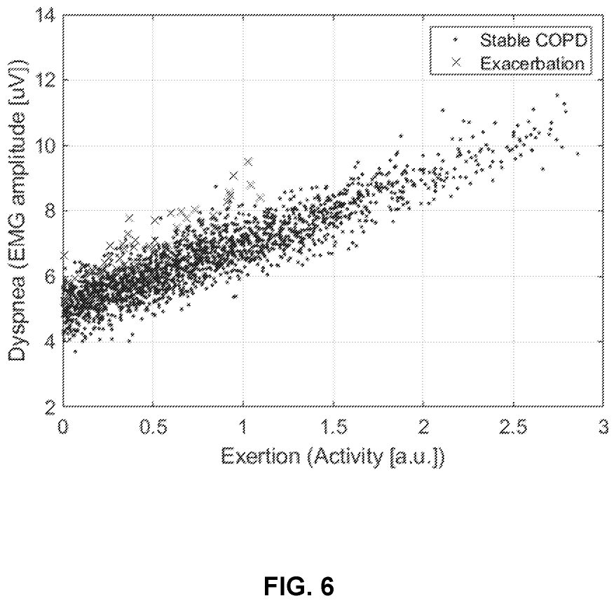

FIG. 6 is a graphical diagram illustrating an example of 2 physiological parameters. While the example of FIG. 6 illustrates 2 physiological parameters, more than 2 physiological parameters may be monitored by IMD 10. In this example, IMD 10 may monitor an EMG of respiratory muscles indicative of dyspnea and an activity count (e.g., from one or more accelerometer signals) indicative of a level of exertion of patient 4. In this example, the EMG of respiratory muscles and the activity count are determined every 15 minutes (˜100×/day). The dots represent the distribution of the parameters of the past month while the health status of patient 4 was stable, whereas the x's represent the data collected during the first day of an acute exacerbation event. As can be seen, the x's are mainly clustered on the top half of or above the dots, particularly when the exertion level is higher. This may be an objective indication of an acute exacerbation event. While the patient may not notice an increase in dyspnea intensity beyond the normal daily variation, IMD 10 considering the dyspnea intensity together with the associated level of exertion, and comparing new data with the data collected over the past month may detect a worsening of dyspnea. For example, IMD 10 may determine statistical properties based of a comparison of the determined physiological parameters to the N-dimensional (two-dimensional in the example of FIG. 6) probability distribution function and calculated a COPD-exacerbation risk score based on the statistical properties. For example, IMD 10 may estimate the joint probability distribution function by fitting an N-dimensional function to the historical data (collected during stable COPD). The probability distribution function may then be used to calculate the probability (e.g., likelihood) of observing the P most recent sets of N physiological variables (with P greater or equal then 1). For example, if every 15 minutes a set of N physiological parameters is determined, P may be 96 to include the data from the preceding 24 hours. This probability (e.g., the likelihood of observing the P most recent N-dimensional sets of physiological parameters) may be used to calculate a risk score for a COPD exacerbation (e.g., the COPD-exacerbation risk score). For example, the COPD-exacerbation risk score may be calculated as the inverse (1/probability) of the probability or 1-the probability itself (e.g., a percentage). In some examples, the COPD-exacerbation risk score may be calculated as the probability itself.

In another example, the P sets of N physiological variables may be grouped according to a categorical variable. For example, the data may be grouped in 4 groups according to the time of day, e.g., night (12 am-6 am), morning (6-12 pm), afternoon (12 pm-6 pm), evening (6 pm-12 pm). For each group, a different probability density function may be estimated based on the historical data, and the probability (e.g., likelihood) of observing the P most recent sets of N physiological variables of each group (with P greater or equal then 1) may be calculated. IMD 10 may then calculate a COPD-exacerbation risk score by combining the probabilities of each group. In this case, P will typically be smaller; in the example where the data is grouped in 4 groups according to time of day, for example, P may be 24.

In another example, the COPD-exacerbation risk score may be directly calculated from the statistical properties of the sets of N physiological variables. For example, IMD 10 may calculated the distance (in the N-dimensional space) from the (N-dimensional) average of the historical data to the newest set of determined N physiological variables, and use that distance to determine a COPD-exacerbation risk score. In another example, IMD 10 may determine a surface (in (N-dimensional space) that encompasses a large fraction (e.g., 95%) of the historical data. The COPD-exacerbation risk score may then be determined from the distance (in the N-dimensional space) of the new set of N physiological variables to that surface.

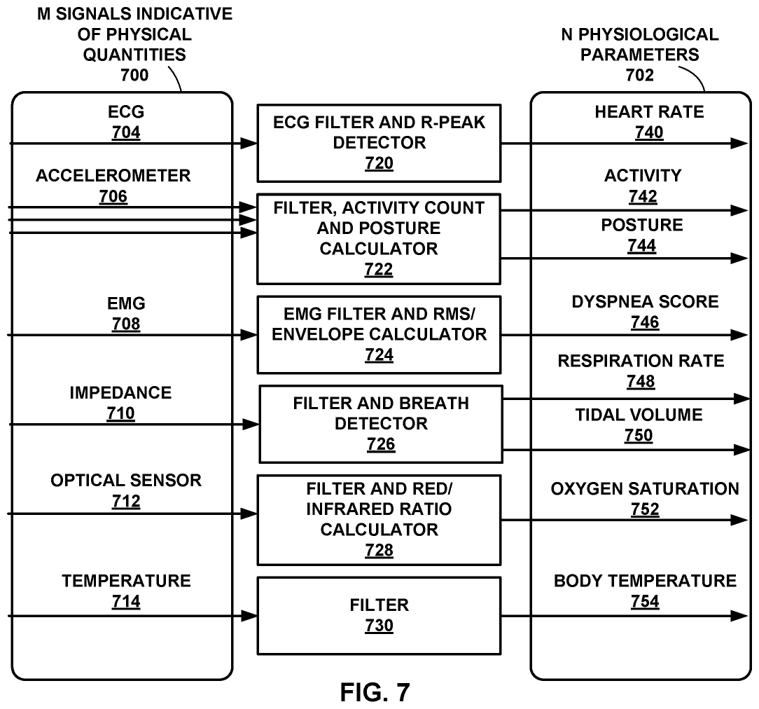

FIG. 7 is a block diagram illustrating example signals indicative of physical quantities, processing, and physiological parameters which may be used to determine an exacerbation. For example, IMD 10 may determine M number of signals indicative of physical quantities 700. IMD 10 may process the M signals indicative of physical quantities 700 to determine N physiological parameters 702. IMD 10 may determine N-dimensional model 62 (FIG. 2), based on N physiological parameters collected over time, for example, one month, which may be stored in historical data 64 (FIG. 2). For example, IMD 10 may generate an N-dimensional probability distribution function or an N-dimensional machine learning model. IMD 10 may determine a COPD-exacerbation risk score by processing the N physiological parameters to N-dimensional model 62. For example, IMD 10 may determine a COPD-exacerbation risk score by comparing the N physiological parameters to N-dimensional model 62. IMD 10 may determine whether the COPD-exacerbation risk score satisfies a threshold 66 (FIG. 2) and generate an indication for output that is based at least in part on the COPD-exacerbation risk score satisfying the threshold. In some examples, the indication may be an alert and IMD 10 may output the alert to external device 12.

For example, one of the M signals indicative of physical quantities 700 may be ECG 704. IMD 10 may process ECG 704, for example by using ECG filter and R-peak detector 720. By filtering and detecting R-peaks in ECG 704, IMD 10 may determine heart rate 740 of patient 4. For example, one of the M signals indicative of physical quantities 700 may be one or more accelerometer signals 706. The example of FIG. 7 includes 3 accelerometer signals, which may correspond to signals from a 3-axis accelerometer. IMD 10 may process one or more accelerometer signals 706, for example, using filter, activity count and posture calculator 722. By filtering, counting activity, calculating posture, and detecting breaths from one or more accelerometer signals 706, ID 10 may determine activity level 742, posture 744, respiration rate 748, and/or tidal volume 750 of patient 4. For example, one of the M signals indicative of physical quantities 700 may be EMG 708. IMD 10 may process EMG 708, for example by using EMG filter and RMS/envelope calculator 724. By filtering and calculating an RMS and envelope of the EMG signal, IMD 10 may determine dyspnea score 746 of patient 4. For example, one of the M signals indicative of physical quantities 700 may be one or more impedance measurements 710. IMD 10 may process one or more impedance measurements 710, for example by using filter and breath detector and/or averager 726. By filtering and detecting breaths and/or averaging impedances, IMD 10 may determine a respiration rate 748, a tidal volume 750, and/or a fluid status 756, of patient 4. For example, one of the M signals indicative of physical quantities 700 may be one or more optical sensor signals 712. IMD 10 may process one or more optical sensor signals 712, for example by using filter and red/infrared ratio calculator 728. By filtering and calculating a red/infrared ratio, IMD 10 may determine an oxygen saturation level of patient 4. For example, one of the M signals indicative of physical quantities 700 may be temperature sensor signal 714. IMD 10 may process temperature sensor signal 714, for example by using filter 730. By filtering temperature sensor signal 714, IMD 10 may determine body temperature 754 of patient 4.

While M signals indicative of physical quantities 700 are set forth, in some examples, fewer signals indicative of physical quantities, additional signals indicative of physical quantities, or different signals indicative of physical quantities may be determined. While N physiological parameters 702 are set forth, in some examples, less physiological parameters, additional physiological parameters, or different physiological parameters may be determined. While each example of processing in FIG. 7 includes filtering, in some examples, filtering may not be a part of the processing.

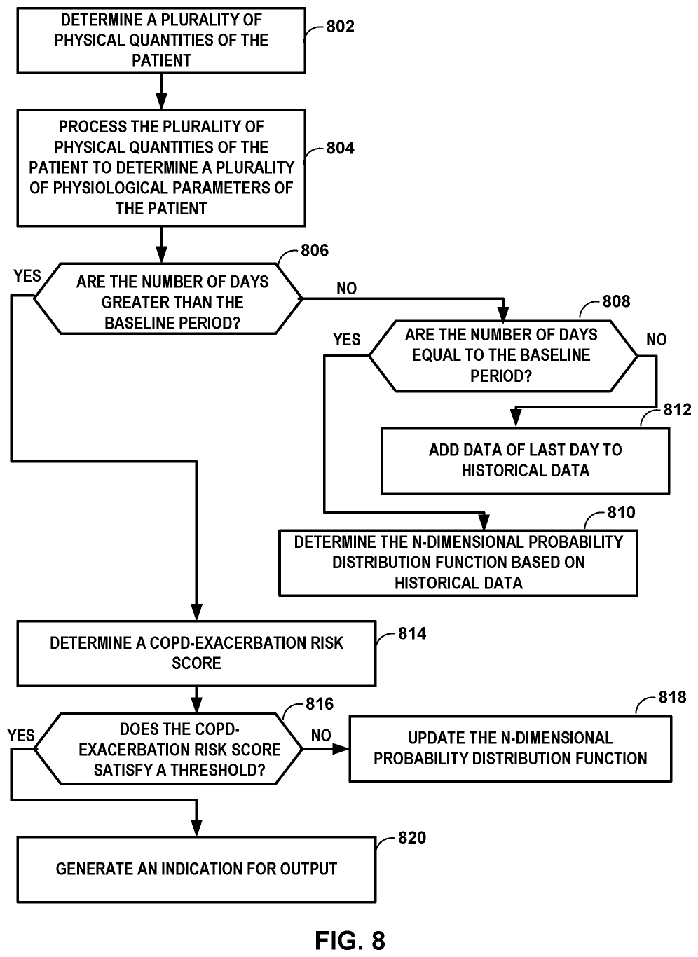

FIG. 8 is a flow diagram illustrating example COPD monitoring techniques of this disclosure. Although described as being performed by IMD 10, the example techniques of FIG. 8 may be performed by any one or more devices described herein, e.g., by processing circuitry of any one or more devices described herein, such as IMD 10, external device 12, and computing system 24.

IMD 10 may determine a plurality of signals indicative of physical quantities of patient 4 (802). For example, IMD 10 may sense or measure a plurality of physical quantities of patient 4 via sensors 58, sensing circuitry 52, and/or processing circuitry 50 (all of FIG. 2). IMD 10 may process the plurality of signals indicative of physical quantities of patient 4 to determine a plurality of physiological parameters of patient 4 (804). For example, IMD 10 may filter or otherwise process M signals indicative of physical quantities 700 to determine N physiological parameters 702 as discussed above with respect to FIG. 7.

In some examples, IMD 10 may determine whether the number of days during which IMD 10 has been determining N physiological parameters 702 is greater than a baseline time period (806). For example, IMD 10 may maintain a count of days or determine a date from communications with external device 12 to determine the number of days during which IMD 10 has been determining N physiological parameters 702. For example, the baseline period may be on the order of one week to one year. If the number of days is not greater than the baseline period (the “NO” path from box 806), IMD 10 may determine whether the number of days during which IMD 10 has been determining N physiological parameters 702 is equal to the baseline period (808). If the number of days during which IMD 10 has been determining N physiological parameters 702 is not equal to the baseline period, IMD 10 may add the determined physiological parameters to historical data 64 (FIG. 2) (812). If the number of days during which IMD 10 has been determining N physiological parameters 702 is equal to the baseline period (the “YES” path from box 808), IMD 10 may determine N-dimensional model 62 (FIG. 2) based on the historical data (810). For example, IMD 10 may determine N-dimensional model 62 based on previously determined physiological parameters of the patient which may be stored in historical data 64.

If the number of days during which IMD 10 has been determining N physiological parameters 702 is greater than the baseline period (the “YES” path from box 806), IMD 10 may determine a COPD-exacerbation risk score (814). For example, IMD 10 may process (e.g., compare) the plurality of physiological parameters of the patient to the N-dimensional model, to determine the COPD-exacerbation risk score. IMD 10 may determine whether the COPD-exacerbation risk score satisfies threshold 66 (FIG. 2) (816). For example, IMD 10 may compare the COPD-exacerbation risk score to a threshold. In some examples, the threshold is predetermined. In other examples, the threshold is dynamic. For example, the IMD 10 may dynamically change the threshold based on a number of determined exacerbations (e.g., a count of the number of times the threshold was met or exceeded), on determined physiological parameters, recent hospitalization, or the like. If the COPD-exacerbation risk score does not satisfy the threshold (the “NO” path from box 816), IMD 10 may update N-dimensional model 62 (818). For example, ID 10 may update N-dimensional model 62 to include the determined plurality of physiological parameters of patient 4 from box 804. If the COPD-exacerbation risk score satisfies the threshold (the “YES” path from box 816), IMD 10 may generate an indication for output (820). In some examples, the indication may be an alert and ID 10 may output the alert to external device 12.

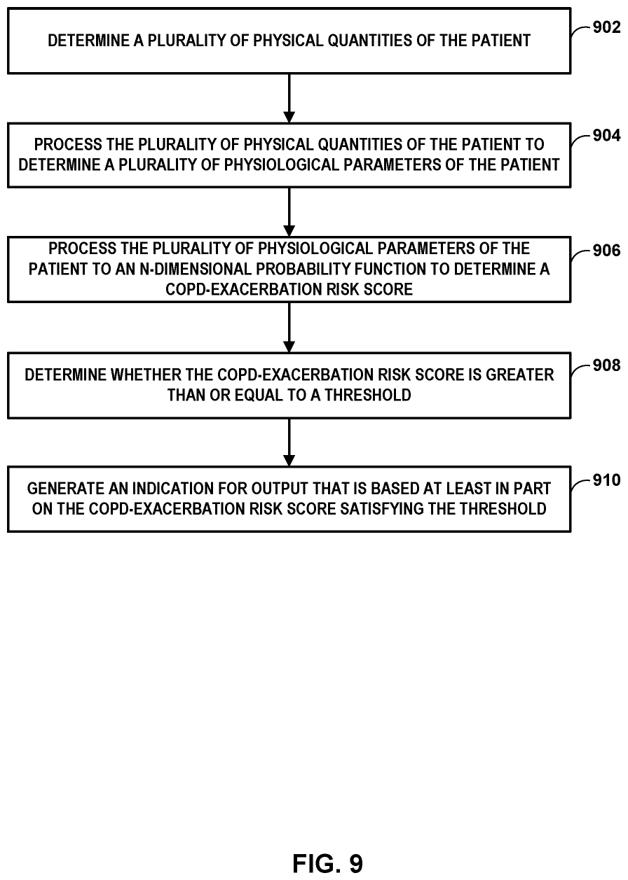

FIG. 9 is a flow diagram illustrating an additional example of COPD monitoring techniques of this disclosure. Although described as being performed by IMD 10, the example techniques of FIG. 8 may be performed by any one or more devices described herein, e.g., by processing circuitry of any one or more devices described herein, such as IMD 10, external device 12, and computing system 24.

IMD 10 may determine a plurality of signals indicative of physical quantities of the patient (902). For example, IMD 10 may sense or measure a plurality of physical quantities of patient 4 via sensors 58, sensing circuitry 52, and/or processing circuitry 50 (all of FIG. 2).

IMD 10 may process the plurality of signals indicative of physical quantities of the patient to determine a plurality of physiological parameters of the patient (904). For example, IMD 10 may filter or otherwise process M signals indicative of physical quantities 700 to determine N physiological parameters 702 as discussed above with respect to FIG. 7.

IMD 10 may process the plurality of physiological parameters of the patient to N-dimensional model 62 (FIG. 2), where N is equal to a count of the plurality of physiological parameters, to determine a COPD-exacerbation risk score (906). For example, IMD 10 may compare the plurality of physiological parameters determined during a given day or time period to N-dimensional model 62. In some examples, IMD 10 may determine N-dimensional model 62 based on previously determined physiological parameters of patient 4 (which may be stored in historical data 64 (FIG. 2)), e.g., during a baseline time period, during the last month, during the lifetime of IMD 10, or the like.

IMD 10 may determine whether the COPD-exacerbation risk score satisfies threshold 66 (FIG. 2) (908). For example, IMD 10 may compare the COPD-exacerbation risk score to the threshold to determine whether the COPD-exacerbation risk score satisfies a threshold. IMD 10 may generate an indication that is based on the COPD-exacerbation risk score satisfying the threshold (910). For example, IMD 10 may generate an alert and wirelessly transmit a signal indicative of the alert via communication circuitry 54 to external device 12. External device 12 may deliver the alert to patient 4, a caregiver of patient 4, or a clinician, enabling patient 4 or the caregiver of patient 4 to seek early treatment or enabling the clinician to administer new or different treatments for the COPD symptoms.

In some examples, IMD 10 may update N-dimensional model 62 to include information indicative of the determined physiological parameters of the patient. In some examples, the plurality of signals indicative of physical quantities includes an ECG and the plurality of physiological parameters include a heart rate, and wherein the heart rate is determined based on the ECG. In some examples, the plurality of signals indicative of physical quantities comprise at least one accelerometer signal and the plurality of physiological parameters comprise at least one of an activity, a posture, a respiration rate, or a tidal volume, and wherein the at least one of the activity, the posture, the respiration rate, or the tidal volume is determined based on the at least one accelerometer signal. In some examples, the plurality of signals indicative of physical quantities includes an EMG and the plurality of physiological parameters include a dyspnea score, and wherein the dyspnea score is determined based on the EMG. In some examples, the plurality of signals indicative of physical quantities comprise an impedance measurement and the plurality of physiological parameters comprise at least one of a respiration rate, a tidal volume, or a fluid status, and wherein the at least one of the respiration rate, the tidal volume or the fluid status is determined based on the impedance measurement.

In some examples, the plurality of signals indicative of physical quantities includes at least one optical sensor signal, and the plurality of physiological parameters include an oxygen saturation level, and wherein the oxygen saturation level is determined based on the at least one optical sensor signal. In some examples, the plurality of signals indicative of physical quantities includes a temperature sensor signal and the plurality of physiological parameters include temperature of the patient, and wherein the temperature of the patient is based on the temperature sensor signal.

By providing an alert when a plurality of physiological parameters indicate that a patient may be experiencing an exacerbation event of COPD symptoms, a medical device may facilitate earlier treatment than otherwise may occur, thereby reducing the need for hospitalization and/or better preserving the health of the patient.

In some examples, the techniques of the disclosure include a system that comprises means to perform any method described herein. In some examples, the techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1. A method comprising: determining, by a medical device, a plurality of signals indicative of physical quantities of the patient; processing, by the processing circuitry, the plurality of signals indicative of physical quantities of the patient to determine a plurality of physiological parameters of the patient; processing, by the processing circuitry, the plurality of physiological parameters of the patient to an N-dimensional model, where N is equal to a count of the plurality of physiological parameters, to determine a chronic obstructive pulmonary disease (COPD)-exacerbation risk score; determining, by the processing circuitry, whether the COPD-exacerbation risk score satisfies a threshold; and generating, by the processing circuitry, an indication for output that is based at least in part on the COPD-exacerbation risk score satisfying the threshold.

Example 2. The method of example 1, further comprising: determining, by the processing circuitry, the N-dimensional model based on previously determined physiological parameters of the patient.

Example 3. The method of example 1 or example 2, further comprising: updating, by the processing circuitry, the N-dimensional model to include information indicative of the determined physiological parameters of the patient.

Example 4. The method of any combination of examples 1-3, wherein the plurality of signals indicative of physical quantities comprise an electrocardiogram and the plurality of physiological parameters comprise a heart rate, and wherein the heart rate is determined based on the electrocardiogram.

Example 5. The method of any combination of examples 1-4, wherein the plurality of signals indicative of physical quantities comprise at least one accelerometer signal and the plurality of physiological parameters comprise at least one of an activity, a posture, a respiration rate, or a tidal volume, and wherein the at least one of the activity, the posture, the respiration rate, or the tidal volume is determined based on the at least one accelerometer signal.

Example 6. The method of any combination of examples 1-5, wherein the plurality of signals indicative of physical quantities comprise an electromyogram and the plurality of physiological parameters comprise a dyspnea score, and wherein the dyspnea score is determined based on the electromyogram.

Example 7. The method of any combination of examples 1-6, wherein the plurality of signals indicative of physical quantities comprise an impedance measurement and the plurality of physiological parameters comprise at least one of a respiration rate, a tidal volume, or a fluid status, and wherein the at least one of the respiration rate, the tidal volume or the fluid status is determined based on the impedance measurement.

Example 8. The method of any combination of examples 1-7, wherein the plurality of signals indicative of physical quantities comprise at least one optical sensor signal and the plurality of physiological parameters comprise an oxygen saturation level, and wherein the oxygen saturation level is determined based on the at least one optical sensor signal.

Example 9. The method of any combination of examples 1-8, wherein the plurality of signals indicative of physical quantities comprise a temperature sensor signal and the plurality of physiological parameters comprise a temperature of the patient, and wherein the temperature of the patient is based on the temperature sensor signal.

Example 10. A medical device system comprising: memory configured to store an N-dimensional model, where N is equal to a count of a plurality of physiological parameters; and processing circuitry communicatively coupled to the memory, the processing circuitry being configured to: determine a plurality of signals indicative of physical quantities of the patient; process the plurality of signals indicative of physical quantities of the patient to determine the plurality of physiological parameters of the patient; process the plurality of physiological parameters of the patient to the N-dimensional model to determine a chronic obstructive pulmonary disease (COPD)-exacerbation risk score; determine whether the COPD-exacerbation risk score satisfies a threshold; and generate an indication for output that is based at least in part on the COPD-exacerbation risk score satisfying the threshold.

Example 11. The medical device of example 10, wherein the processing circuitry is further configured to: determine the N-dimensional model based on previously determined physiological parameters of the patient.

Example 12. The medical device of example 10 example 11, wherein the processing circuitry is further configured to: update the N-dimensional model to include information indicative of the determined physiological parameters of the patient.

Example 13. The medical device of any combination of examples 10-12, wherein the plurality of signals indicative of physical quantities comprise an electrocardiogram and the plurality of physiological parameters comprise a heart rate, and wherein the heart rate is determined based on the electrocardiogram.

Example 14. The medical device of any combination of examples 10-13, wherein the plurality of signals indicative of physical quantities comprise at least one accelerometer signal and the plurality of physiological parameters comprise at least one of an activity, a posture, a respiration rate, or a tidal volume, and wherein the at least one of the activity, the posture, respiration rate, or tidal volume is determined based on the at least one accelerometer signal.

Example 15. The medical device of any combination of examples 10-14, wherein the plurality of signals indicative of physical quantities comprise an electromyogram and the plurality of physiological parameters comprise a dyspnea score, and wherein the dyspnea score is determined based on the electromyogram.

Example 16. The medical device of any combination of examples 10-15, wherein the plurality of signals indicative of physical quantities comprise an impedance measurement and the plurality of physiological parameters comprise at least one of a respiration rate, a tidal volume, or a fluid status, and wherein the at least one of the respiration rate, the tidal volume or the fluid status is determined based on the impedance measurement.

Example 17. The medical device of any combination of examples 10-16, wherein the plurality of signals indicative of physical quantities comprise at least one optical sensor signal and the plurality of physiological parameters comprise an oxygen saturation level, and wherein the oxygen saturation level is determined based on the at least one optical sensor signal.

Example 18. The medical device of any combination of examples 10-17, wherein the plurality of signals indicative of physical quantities comprise a temperature sensor signal and the plurality of physiological parameters comprise a temperature of the patient, and wherein the temperature of the patient is based on the temperature sensor signal.

Example 19. The medical device of any combination of examples 10-18, comprising an implantable medical device or an insertable medical device.

Example 20. A non-transitory computer-readable storage medium storing instructions, which, when executed, cause processing circuitry to: determine a plurality of signals indicative of physical quantities of a patient; process the plurality of signals indicative of physical quantities of the patient to determine a plurality of physiological parameters of the patient; process the plurality of physiological parameters of the patient to the N-dimensional model to determine a chronic obstructive pulmonary disease (COPD)-exacerbation risk score, where N is equal to a count of the plurality of physiological parameters; determine whether the COPD-exacerbation risk score satisfies a threshold; and generate an indication for output that is based at least in part on the COPD-exacerbation risk score satisfying the threshold.

Various examples have been described. These and other examples are within the scope of the following claims.

1. A medical device system comprising:
memory configured to store an N-dimensional model, where N is equal to a count of a plurality of physiological parameters; and
processing circuitry communicatively coupled to the memory, the processing circuitry being configured to:
determine a plurality of signals indicative of physical quantities of the patient;
process the plurality of signals indicative of physical quantities of the patient to determine the plurality of physiological parameters of the patient;
compare the plurality of physiological parameters of the patient to the N-dimensional model to determine a chronic obstructive pulmonary disease (COPD)-exacerbation risk score;
determine whether the COPD-exacerbation risk score satisfies a threshold; and
generate an indication for output that is based at least in part on the COPD-exacerbation risk score satisfying the threshold.

2. The medical device of claim 1, wherein the processing circuitry is further configured to determine the N-dimensional model based on previously determined physiological parameters of the patient.

3. The medical device of claim 1, wherein the processing circuitry is further configured to update the N-dimensional model to include information indicative of the determined physiological parameters of the patient.

4. The medical device of claim 1, wherein the plurality of signals indicative of physical quantities comprise an electrocardiogram and the plurality of physiological parameters comprise a heart rate, and wherein the heart rate is determined based on the electrocardiogram.

5. The medical device of claim 1, wherein the plurality of signals indicative of physical quantities comprise at least one accelerometer signal and the plurality of physiological parameters comprise at least one of an activity, a posture, a respiration rate, or a tidal volume, and wherein the at least one of the activity, the posture, respiration rate, or tidal volume is determined based on the at least one accelerometer signal.

6. The medical device of claim 1, wherein the plurality of signals indicative of physical quantities comprise an electromyogram and the plurality of physiological parameters comprise a dyspnea score, and wherein the dyspnea score is determined based on the electromyogram.

7. The medical device of claim 1, wherein the plurality of signals indicative of physical quantities comprise an impedance measurement and the plurality of physiological parameters comprise at least one of a respiration rate, a tidal volume, or a fluid status, and wherein the at least one of the respiration rate, the tidal volume or the fluid status is determined based on the impedance measurement.

8. The medical device of claim 1, wherein the plurality of signals indicative of physical quantities comprise at least one optical sensor signal and the plurality of physiological parameters comprise an oxygen saturation level, and wherein the oxygen saturation level is determined based on the at least one optical sensor signal.

9. The medical device of claim 1, wherein the plurality of signals indicative of physical quantities comprise a temperature sensor signal and the plurality of physiological parameters comprise a temperature of the patient, and wherein the temperature of the patient is based on the temperature sensor signal.

10. The medical device of claim 1, comprising an implantable medical device or an insertable medical device.

11. A method for controlling operation of processing circuitry of a medical system, the method comprising:
determining, by a medical device, a plurality of signals indicative of physical quantities of the patient;
processing, by the processing circuitry, the plurality of signals indicative of physical quantities of the patient to determine a plurality of physiological parameters of the patient;
comparing, by the processing circuitry, the plurality of physiological parameters of the patient to an N-dimensional model, where N is equal to a count of the plurality of physiological parameters, to determine a chronic obstructive pulmonary disease (COPD)-exacerbation risk score;
determining, by the processing circuitry, whether the COPD-exacerbation risk score satisfies a threshold; and
generating, by the processing circuitry, an indication for output that is based at least in part on the COPD-exacerbation risk score satisfying the threshold.

12. The method of claim 11, further comprising:
determining, by the processing circuitry, the N-dimensional model based on previously determined physiological parameters of the patient.

13. The method of claim 11, further comprising:
updating, by the processing circuitry, the N-dimensional model to include information indicative of the determined physiological parameters of the patient.

14. The method of claim 11, wherein the plurality of signals indicative of physical quantities comprise an electrocardiogram and the plurality of physiological parameters comprise a heart rate, and wherein the heart rate is determined based on the electrocardiogram.

15. A non-transitory computer-readable storage medium storing instructions, which, when executed, cause processing circuitry to:
determine a plurality of signals indicative of physical quantities of a patient;
process the plurality of signals indicative of physical quantities of the patient to determine a plurality of physiological parameters of the patient;
compare the plurality of physiological parameters of the patient to the N-dimensional model to determine a chronic obstructive pulmonary disease (COPD)-exacerbation risk score, where N is equal to a count of the plurality of physiological parameters;
determine whether the COPD-exacerbation risk score satisfies a threshold; and
generate an indication for output that is based at least in part on the COPD-exacerbation risk score satisfying the threshold.

16. The method of claim 11, wherein the plurality of signals indicative of physical quantities comprise at least one accelerometer signal and the plurality of physiological parameters comprise at least one of an activity, a posture, a respiration rate, or a tidal volume, and wherein the at least one of the activity, the posture, respiration rate, or tidal volume is determined based on the at least one accelerometer signal.

17. The method of claim 11, wherein the plurality of signals indicative of physical quantities comprise an electromyogram and the plurality of physiological parameters comprise a dyspnea score, and wherein the dyspnea score is determined based on the electromyogram.

18. The method of claim 11, wherein the plurality of signals indicative of physical quantities comprise an impedance measurement and the plurality of physiological parameters comprise at least one of a respiration rate, a tidal volume, or a fluid status, and wherein the at least one of the respiration rate, the tidal volume or the fluid status is determined based on the impedance measurement.

19. The method of claim 11, wherein the plurality of signals indicative of physical quantities comprise at least one optical sensor signal and the plurality of physiological parameters comprise an oxygen saturation level, and wherein the oxygen saturation level is determined based on the at least one optical sensor signal.

20. The method of claim 11, wherein the plurality of signals indicative of physical quantities comprise a temperature sensor signal and the plurality of physiological parameters comprise a temperature of the patient, and wherein the temperature of the patient is based on the temperature sensor signal.
